(19) United States
(12) Patent Application Publication
(10) Pub. No.: US 2026/0335259 A1
(43) Pub. Date: Oct. 29, 2026

(54) TOPICAL COMPOSITION FOR TREATING PAIN AND INFLAMMATION

(71) Applicants: Amol Soin, Dayton, OH (US); Aviraj Gabriel Soin, Dayton, OH (US); Dhilen Noah Soin, Dayton, OH (US); Brijen David Soin, Dayton, OH (US)

(72) Inventors: Amol Soin, Dayton, OH (US); Aviraj Gabriel Soin, Dayton, OH (US); Dhilen Noah Soin, Dayton, OH (US); Brijen David Soin, Dayton, OH (US)

(21) Appl. No.: 19/539,818

(22) Filed: Feb. 13, 2026

Related U.S. Application Data
(60) Provisional application No. 63/760,460, filed on Feb. 19, 2025.

(51) Int. Cl. A61K 31/16; A61K 9/00; A61K 9/06; A61K 47/10; A61K 47/26; A61K 47/36

(52) U.S. Cl. CPC A61K 31/16; A61K 9/0014; A61K 9/06; A61K 47/10; A61K 47/26; A61K 47/36

(57) ABSTRACT

A topical composition comprising plant-based active ingredients for the treatment of pain and inflammatory conditions in mammals. An aspect of the topical composition comprises hydroxy-α-sanshool, spilanthol, and one or more topically acceptable excipients.

### CROSS-REFERENCE TO RELATED APPLICATIONS

This application claims the benefit of and priority to U.S. Provisional Application No. 63/760,460, filed Feb. 19, 2025, which application is incorporated herein by reference in its entirety.

### TECHNICAL FIELD OF THE INVENTION

The present disclosure relates to the field of pharmaceuticals, specifically to a topical composition comprising plant-based active ingredients for the treatment of pain and inflammatory conditions in mammals.

### BACKGROUND

An active area of research focuses on developing safer and more effective methods for alleviating pain through topical formulations. Over the years, various topical pain relief formulations, including lotions and ointments with synthetic anti-inflammatory agents, have been introduced. However, many of these treatments offer limited efficacy or provide relief that lasts only for a short duration. While existing formulations deliver varying degrees of pain relief, there is a growing demand for innovative solutions that incorporate plant-based active ingredients, offering convenient and longer-lasting pain relief. The present disclosure satisfies these needs and provides further related advantages.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Aspects of the present disclosure relate to a topical composition comprising hydroxy-α-sanshool and spilanthol as active ingredients for the treatment of pain and inflammatory conditions in mammals. This composition delivers hydroxy-α-sanshool and spilanthol in amounts effective for alleviating pain. It enables topical application to the afflicted area, allowing the active ingredients to be absorbed through the skin or oral mucosa, thereby providing pain relief. The disclosed composition offers convenient, longer-lasting therapy for painful inflammatory conditions, including arthritis, fibromyalgia, neuropathic pain, toothache, and chronic pain.

In various embodiments, such a topical composition includes, based on 100 weight/weight percent, from about 0.001% to about 20% by weight of hydroxy-α-sanshool, from about 0.001% to about 20% by weight of spilanthol, and one or more topically acceptable excipients. The composition may be formulated as an ointment, cream, gel, patch, lotion, spray, or foam to suit different application preferences.

In an aspect, the present invention relates to a topical composition for treating pain and inflammation, wherein the composition comprising:
(a) from about 0.001 wt % to about 20 wt % hydroxy-α-sanshool based on the total weight of the composition;
(b) from about 0.001 wt % to about 20 wt % spilanthol based on the total weight of the composition; and,
(c) from about 5 wt % to about 95 wt % of one or more topically acceptable excipients based on the total weight of the composition.

In another aspect of the present invention, the one or more topically acceptable excipients of the composition is selected from the group consisting of gel-forming agents, permeation enhancers, solubilizers, antioxidants, hydrating agents, aqueous carriers, preservatives, pH adjusters, pH buffering agents, sweeteners, flavoring agents, bio-adhesive enhancers, encapsulating agents, and release control agents.

In yet another aspect of the present invention, the gel-forming agents of the composition are selected from the group consisting of polyacrylates, cellulose derivatives, polyvinyl alcohols, polyvinyl acetate, and mixtures thereof.

In yet another aspect of the present invention, the permeation enhancers of the composition are selected from the group consisting of glycols, polyols, fatty acids, ether alcohols, cyclic ether alcohols, alkoxy substituted cyclic ethers, fatty alcohol esters, and mixtures thereof.

In yet another aspect of the present invention, the solubilizers can be ethanol, Polysorbate 80, or mixtures thereof.

In yet another aspect of the present invention, the antioxidants can be selected from the group consisting of tocopherol, butyl hydroxytoluene, ascorbyl palmitate, propyl gallate, sodium metabisulfite, potassium metabisulfite and mixtures thereof.

In yet another aspect of the present invention, the antioxidants are hydrating agents glycerin, hyaluronic acid, petrolatum, cetostearyl alcohol or mixture thereof, or the aqueous carriers is water.

In yet another aspect of the present invention, the preservatives are selected from the group consisting of phenoxyethanol, parabens, sodium benzoate, potassium sorbate, thimerosal, chlorobutanol, benzalkonium chloride, benzyl alcohol and mixtures thereof.

In yet another aspect of the present invention, the pH adjusters is selected from the group consisting of citric acid, lactic acid, sodium hydroxide, and triethanolamine; or the pH buffering agents is selected from the group consisting of citric acid, sodium citrate, lactic acid, sodium lactate, succinic acid, sodium succinate, sodium acetate, acetic acid, sodium phosphate, phosphoric acid, sodium ascorbate, tartaric acid, maleic acid, ascorbic acid, sodium bicarbonate, histidine, sodium benzoate, benzoic acid, and combinations thereof.

In yet another aspect of the present invention, the pH sweeteners can be selected from the group consisting of xylitol, aspartame, dextrose, mannitol, saccharin sodium, sorbitol, sucrose, and combinations thereof; or the flavoring agent is selected from the group consisting of peppermint oil, anise oil, cinnamon oil, cocoa, menthol, orange oil, vanillin and combination thereof.

In another aspect of the present invention, the bio-adhesive enhancers is chitosan or trimethyl chitosan chloride.

In another aspect of the present invention, the encapsulating agents are selected from the group consisting of ethyl cellulose, methyl ethyl cellulose, methyl cellulose, hydroxypropylmethyl cellulose, alginates, and combinations thereof.

In another aspect of the present invention, the release control agents are selected from the group consisting of hydroxypropyl cellulose (HPC), hydroxypropyl methyl cellulose (HPMC), polyvinyl alcohol (PVA), Carbopol, polyethylene oxide (PEO), and combination thereof.

In another aspect of the present invention, the composition is in the form of ointment, cream, gel, patch, lotion, spray, or foam.

In another aspect of the present invention, the composition is controlled release formulation, or sustained release formulation.

In another aspect of the present invention, the composition is skin topical gel formulation or oral mucosal gel formulation.

In another aspect, the present invention relates to a method of treating a pain or inflammation comprising administering a therapeutically effective amount of the composition of present invention to a subject in need thereof.

In another aspect of the present invention, the composition comprises, based on the total weight of the composition:
(a) from about 0.001 wt % to about 20 wt % hydroxy-α-sanshool;
(b) from about 0.001 wt % to about 20 wt % spilanthol;
(c) from about 0.1 wt % to about 10 wt % of one or more gel-forming agents;
(d) from about 0.1 wt % to about 15 wt % of one or more permeation enhancers;
(e) from about 0.5 wt % to about 25% of one or more solubilizers;
(f) from about 1 wt % to about 8 wt % of one or more hydrating agents;
(g) from about 0.1 wt % to about 5 wt % of one or more preservatives;
(h) from about 0.05 wt % to about 5 wt % of a pH adjuster;
(i) from about 0.05 wt % to about 3 wt % of a pH buffering agent; and
(j) from about 1 wt % to about 80 wt % water.

In another aspect of the present invention, the composition further comprises, based on the total weight of the composition:
(a) from about 0.1 wt % to about 5 wt % of one or more antioxidants; or
(b) from about 0.5 wt % to about 5 wt % of an encapsulating agent; or
(c) from about 0.1 wt % to about 10 wt % of one or more release control agents; or
(d) from about 0.1 wt % to about 8 wt % of bio-adhesive enhancers; or
(e) from about 0.5 wt % to about 10 wt % of one or more sweeteners; or
(f) from about 0.1 wt % to about 5 wt % of one or more flavoring agents.

The present disclosure is also directed to methods for treating pain by topically administering a composition containing hydroxy-α-sanshool and spilanthol to an area in need of pain relief.

Various features, objects, aspects and advantages of the inventive subject matter will become more apparent from the following detailed description of various embodiments.

### BRIEF DESCRIPTION OF THE DRAWINGS

FIG. 1 shows Simulated In Vitro Release (Skin Application) of the present invention; and,

FIG. 2 shows Simulated In Vitro Release (Oral Mucosal Application) of the present invention.

### DETAILED DESCRIPTION OF THE INVENTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless otherwise noted, all measurements are in standard metric units.

Unless otherwise noted, all instances of the words “a,” “an,” or “the” can refer to one or more than one of the word(s) that they modify.

Unless otherwise noted, the phrase “at least one of” means one or more than one of an object(s).

The term “about” as used herein contemplates what is ordinarily meant in the art, and includes an acceptable range for a particular value as determined by a skilled artisan in the art. This, in turn, may depend on how the value is determined, or it may depend on the result to be achieved. For example, used in the context of chemical compositions, the term “about” allows for a degree of variation inherent in the methods utilized for measuring the chemical compounds, and/or also allows for a degree of variation that produces the same or similar result in term of desired effectiveness. In some embodiments, the term “about” when used before a numerical value indicates that the value may vary within reasonable range, such as ±0.5%, ±0.2%, and ±0.1%. When “about” is used before a pH value, it indicates that the pH value may vary ±0.2, ±0.1, ±0.05 or ±0.02 pH units.

Unless otherwise noted, when a range of numbers refers to numbers that are easily and routinely varied in a laboratory setting, such as weight, mass, concentration, temperature, or pressure, then the range of numbers or measurements includes all numbers in that range. For example, the range of 1 mg to 10 mg includes 2.0, 3.0, 4.0, 5.0, 6.0, 7.0, 8.0, 9.0 and any sub range/number therein.

Unless otherwise noted, the term “for example” or “e.g.,” as used herein, is used merely by way of example, and should not be construed as limiting the present disclosure to only those items explicitly referred to in the specification.

Unless otherwise noted, all reactions, procedures, and formulations were or can be performed under normal temperature and pressure.

As used herein, the term “topical” refers to the application of the composition described in this disclosure to the skin or oral mucosal surface. This includes direct application or indirect application when the composition is impregnated onto a carrier intended for contact with the skin or oral mucosa, such as a patch, paper, wipe, textile, or transdermal device.

Unless otherwise noted, the term “plant-based” refers to a compound or substance that is found in a plant in nature, whether being extracted directly from a plant, or that is synthetically produced to have the equivalent molecular structure or configuration as the compound or substance as it exists in nature.

As used herein, the term “mammal” refers to members of the class Mammalia, encompassing higher vertebrates, including but not limited to humans.

The term “effective amount” refers to a quantity of a compound (e.g., a compound described in the present disclosure) sufficient to achieve beneficial or desired results. This amount may be administered in one or multiple applications, dosages, or administrations.

As used herein, the term “treating” includes any effect, e.g., lessening, reducing, modulating, ameliorating or eliminating, that results in the improvement of the condition, disease, disorder, and the like, or ameliorating a symptom thereof.

As used herein, the phrase “topically acceptable excipient” refers to an excipient that is suitable for topical application, non-toxic, non-irritating to the skin and/or oral mucous membranes, does not induce an allergic response, and is chemically stable.

Hydroxy-α-sanshool, a bioactive compound from Sichuan pepper, shows potential for pain management due to its unique interaction with the nervous system. It targets transient receptor potential (TRP) channels, specifically TRPV1 and TRPA1, which are involved in detecting pain and irritants like heat and cold. By activating these channels, hydroxy-α-sanshool desensitizes nerve fibers, reducing their sensitivity to painful stimuli. Additionally, it affects sensory nerve endings, inducing a tingling sensation that may mask pain. It also modulates mechanosensitive Piezo channels, reducing hypersensitivity to mechanical pain, such as in neuropathy or inflammation. Prolonged exposure may lead to temporary desensitization, offering potential for acute pain relief. Hydroxy-α-sanshool can be used for treating neuropathic and chronic pain, as well as in topical analgesic formulations, providing localized pain relief without systemic side effects.

Spilanthol, a bioactive compound from the plant Acmella oleracea, is recognized for its analgesic properties. It exerts a local anesthetic effect by desensitizing nerve endings, providing temporary pain relief, particularly for oral conditions like toothaches. Spilanthol also demonstrates anti-inflammatory effects by reducing the production of inflammatory cytokines and enzymes such as COX-2, helping to alleviate pain linked to inflammation. Additionally, it modulates nociceptive pathways by affecting pain-related receptors and ion channels, reducing sensitivity to painful stimuli. These mechanisms make spilanthol effective for oral pain relief and, with ongoing research, it is being explored for broader pain management applications.

Embodiments herein are directed to a topical composition comprising hydroxy-α-sanshool and spilanthol as active ingredients for treating pain and inflammatory conditions in mammals. The composition is designed to deliver effective amounts of these active ingredients to alleviate pain. It can be applied topically to the affected area, where the ingredients are absorbed through the skin or oral mucosa, providing relief from pain. This composition offers a convenient, long-lasting solution for managing painful inflammatory conditions, such as arthritis, fibromyalgia, neuropathic pain, toothaches, and chronic pain.

In various embodiments, such a topical composition includes, based on 100 weight/weight percent, from about 0.001% to about 20% by weight of hydroxy-α-sanshool, from about 0.001% to about 20% by weight of spilanthol, and one or more topically acceptable excipients. The one or more topically acceptable excipients are not particularly limited and may be appropriately selected depending on the intended purpose. Examples of topically acceptable excipients include, but not limited to, gel-forming agents, permeation enhancers, viscosity enhancers, mucosal absorption enhancers, solubilizers, stabilizers, antioxidants, hydrating agents, aqueous carriers, preservatives, pH adjusters, pH buffering agents, sweeteners, flavoring agents, bio-adhesive enhancers, encapsulating agents, and release control agents. Such excipients may be employed in amounts of about 5% to about 95%, preferably about 10% to about 80% of the total weight of the topical composition.

Some embodiments herein provide a composition for topical application to a mammal's skin for pain relief. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more gel-forming agents | 0.1% to 10% | 0.5% to 7% |
| One or more permeation enhancers | 0.1% to 15% | 0.5% to 12% |
| One or more solubilizers | 0.5% to 25% | 0.5% to 15% |
| One or more hydrating agents | 1% to 8% | 2% to 5% |
| Water | 1% to 80% | 10% to 70% |

Some embodiments herein provide a composition for topical application to a mammal's skin for pain relief. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more gel-forming agents | 0.1% to 10% | 0.5% to 7% |
| One or more permeation enhancers | 0.1% to 15% | 0.5% to 12% |
| One or more solubilizers | 0.5% to 25% | 0.5% to 15% |
| One or more antioxidants | 0.1% to 5% | 0.1% to 2% |
| One or more hydrating agents | 1% to 8% | 2% to 5% |
| One or more preservatives | 0.1% to 5% | 0.5% to 1% |
| A pH adjuster | 0.05% to 5% | 0.05% to 3% |
| A pH buffering agent | 0.05% to 3% | 0.05% to 2% |
| Water | 1% to 80% | 10% to 70% |

Some embodiments herein provide a composition for topical application to a mammal's oral mucosal surface for treating pain. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more gel-forming agents | 0.1% to 10% | 0.5% to 7% |
| One or more permeation enhancers | 0.1% to 15% | 0.5% to 12% |
| One or more solubilizers | 0.5% to 25% | 0.5% to 10% |
| One or more bio-adhesive enhancers | 0.1% to 8% | 0.5% to 5% |
| One or more sweeteners | 0.5% to 10% | 0.5% to 5% |
| One or more flavoring agents | 0.1% to 5% | 0.1% to 2% |
| One or more hydrating agents | 1% to 8% | 2% to 5% |
| Water | 1% to 80% | 10% to 70% |

Some embodiments herein provide a composition for topical application to a mammal's oral mucosal surface for treating pain. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more gel-forming agents | 0.1% to 10% | 0.5% to 7% |
| One or more permeation enhancers | 0.1% to 15% | 0.5% to 12% |
| One or more solubilizers | 0.5% to 25% | 0.5% to 10% |
| One or more bio-adhesive enhancers | 0.1% to 8% | 0.5% to 5% |
| One or more sweeteners | 0.5% to 10% | 0.5% to 5% |
| One or more flavoring agents | 0.1% to 5% | 0.1% to 2% |
| One or more hydrating agents | 1% to 8% | 2% to 5% |
| One or more preservatives | 0.1% to 5% | 0.5% to 1% |
| A pH adjuster | 0.05% to 5% | 0.05% to 3% |
| A pH buffering agent | 0.05% to 3% | 0.05% to 2% |
| Water | 1% to 80% | 10% to 70% |

Some embodiments herein provide a time-controlled, sustained release topical composition for application to a mammal's skin for the treatment of pain. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more release control agents | 0.1% to 10% | 0.5% to 7% |
| An encapsulating agent | 0.5% to 5% | 1% to 2% |
| One or more permeation enhancers | 0.1% to 15% | 0.5% to 12% |
| One or more solubilizers | 0.5% to 25% | 0.5% to 15% |
| One or more hydrating agents | 1% to 8% | 2% to 5% |
| Water | 1% to 80% | 10% to 70% |

Some embodiments herein provide a time-controlled, sustained release topical composition for application to a mammal's skin for the treatment of pain. The composition has a pH in the range of 4.5 to 6.5 and comprises the following ingredients:

| | Range | Preferred range |
|---|---|---|
| Ingredient | (wt. %) | (wt. %) |
| Hydroxy-α-sanshool | 0.001% to 20% | 0.1% to 10% |
| Spilanthol | 0.001% to 20% | 0.1% to 10% |
| One or more release control agents | 0.1% to 10% | 0.5% to 7% |
| An encapsulating agent | 0.5% to 5% | 1% to 2% |
| One or more permeation enhancers | 0.1% to 15% | 0.5% to 12% |
| One or more solubilizers | 0.5% to 25% | 0.5% to 15% |
| One or more antioxidants | 0.1% to 5% | 0.1% to 2% |
| One or more hydrating agents | 1% to 8% | 2% to 5% |
| One or more preservatives | 0.1% to 5% | 0.5% to 1% |
| A pH adjuster | 0.05% to 5% | 0.05% to 3% |
| A pH buffering agent | 0.05% to 3% | 0.05% to 2% |
| Water | 1% to 80% | 10% to 70% |

In another embodiment, the composition of present invention is a Skin Topical Gel Formulation comprising:

| Ingredient | % (w/w) |
|---|---|
| Hydroxy-α-sanshool | 2.0% |
| Spilanthol | 2.0% |
| Carbomer (polyacrylate) | 1.0% |
| Propylene glycol | 8.0% |
| Ethanol | 8.0% |
| Tocopherol (Vitamin E) | 0.2% |
| Sodium metabisulfite | 0.1% |
| Glycerin | 4.0% |
| Phenoxyethanol | 0.8% |
| Triethanolamine (TEA) | 0.5% |
| Citrate buffer (pH 5-6) | 1.0% |
| Purified water | 72.4% |
| | 100% |

In another embodiment, the composition of present invention is an oral topical Gel Formulation comprising:

| Ingredient | % (w/w) |
|---|---|
| Hydroxy-α-sanshool | 2.0% |
| Spilanthol | 2.0% |
| Carbomer 934P | 1.0% |
| Hydroxypropyl | 2.0% |
| methylcellulose (HPMC) | |
| Propylene glycol | 8.0% |
| Oleic acid | 1.0% |
| Polysorbate 80 | 0.5% |
| Chitosan | 1.0% |
| Xylitol | 2.0% |
| Peppermint oil | 0.2% |
| Glycerin | 4.0% |
| Potassium sorbate | 0.2% |
| Citric acid | 0.1% |
| Sodium citrate buffer | 1.0% |
| Purified water | 75.0% |
| | 100% |

In another embodiment, the composition of present invention is controlled-release gel formulation comprising:

| Ingredient | % (w/w) |
|---|---|
| Hydroxy-α-sanshool | 2.0% |
| Spilanthol | 2.0% |
| Carbopol 934P (Carbomer) | 1.0% |
| Hydroxypropyl | 3.0% |
| methylcellulose (HPMC) | |
| Ethyl cellulose | 1.5% |
| Propylene glycol | 8.0% |
| Oleic acid | 1.0% |
| Polysorbate 80 | 0.5% |
| Glycerin | 4.0% |
| Tocopherol (Vitamin E) | 0.2% |
| Sodium metabisulfite | 0.1% |
| Phenoxyethanol | 0.8% |
| Triethanolamine | 0.5% |
| Citrate buffer (pH ~5.5) | 1.0% |
| Purified water | 74.4% |
| | 100% |

The topical composition disclosed herein incorporates one or more synthetic gel-forming agents to create a structured, smooth, and transparent gel matrix that facilitates the effective delivery and retention of active ingredients. These gel-forming agents, which include polymers such as polyacrylates, cellulose derivatives, polyvinyl alcohols, polyvinyl acetate, and their mixtures, serve as the backbone of the formulation. They impart critical properties such as consistency, stability, and a uniform dispersion of the active ingredients throughout the gel, ensuring consistent therapeutic efficacy upon application. The selection and combination of gel-forming agents can be tailored based on the intended application and specific therapeutic goals. For instance, in formulations requiring long-lasting effects, a combination of Carbomer and HPMC may be used to create a gel matrix. For formulations where rapid initial release followed by sustained effects is desired, blending Carbomer with PVA or cellulose derivatives can achieve the appropriate release kinetics. In some embodiments, Carbomer is used as the gel-forming agent. In some embodiments, Carbomer and hydroxypropyl methylcellulose (HPMC) are used as gel forming agents. Gel-forming agents may be employed in amounts ranging from about 0.1% to 10% by weight, based on the total weight of the topical composition.

The topical composition described herein includes one or more permeation enhancers that enhance the permeability of the lipophilic active ingredients, hydroxy-α-sanshool and spilanthol, through the skin. When formulated for topical application to oral mucosal surface, these permeation enhancers facilitate the penetration of hydroxy-α-sanshool and spilanthol across the buccal mucosa by disrupting the mucosal lipid bilayers, thereby improving the absorption of the active ingredients. Exemplary permeation enhancers include glycols, polyols, fatty acids, ether alcohols, cyclic ether alcohols, alkoxy substituted cyclic ethers, fatty alcohol esters, and mixtures thereof. In some embodiments, propylene glycol is used as the permeation enhancer. In some embodiments, propylene glycol and oleic acid are used as the permeation enhancers. Permeation enhancers may be employed in amounts ranging from about 0.1% to 15% by weight, based on the total weight of the topical composition.

The topical composition described herein includes one or more solubilizers, which are preferably ethanol, Polysorbate 80, or mixtures thereof. These solubilizers enhance the solubility of hydroxy-α-sanshool and spilanthol, both of which are lipophilic compounds, within the composition. Solubilizers may be employed in amounts ranging from about 0.5% to 25% by weight, based on the total weight of the topical composition.

In some embodiments, the topical composition includes one or more antioxidants to prevent the active ingredients, hydroxy-α-sanshool and spilanthol, from oxidative degradation. Examples of antioxidants include tocopherol, butyl hydroxytoluene, ascorbyl palmitate, propyl gallate, sodium metabisulfite, potassium metabisulfite and other topically acceptable antioxidants. In some embodiments, the topical composition includes tocopherol and sodium metabisulfite as the antioxidants. Antioxidants may be employed in amounts ranging from about 0.1% to 5% by weight, based on the total weight of the topical composition.

The topical composition described herein includes one or more hydrating agents or moisturizers, such as, for example, glycerin, hyaluronic acid, petrolatum, cetostearyl alcohol. In some embodiments, glycerin is used as the hydrating agent. The hydrating agent may be employed in amounts ranging from about 1% to 8% by weight, based on the total weight of the topical composition.

A preservative may be employed to increase the shelf-life of the topical composition disclosed herein. A number of well-known and pharmaceutically acceptable preservatives may be used in the present topical composition, including, but not limited to, phenoxyethanol, parabens, sodium benzoate, potassium sorbate, thimerosal, chlorobutanol, benzalkonium chloride, benzyl alcohol and combinations thereof. Phenoxyethanol and potassium sorbate are the preferred preservatives. Preservative may be employed in amounts ranging from about 0.1% to 5% by weight, based on the total weight of the topical composition.

When formulated for topical application to oral mucosal surface, the composition may include one or more flavoring agents and sweeteners to enhance its sensory appeal and user compliance. Examples of flavoring agents include, but are not limited to, peppermint oil, anise oil, cinnamon oil, cocoa, menthol, orange oil, and vanillin. Flavoring agents can be incorporated in amounts ranging from about 0.1% to about 5% by weight of the total composition. Similarly, examples of sweeteners include, but are not limited to, xylitol, aspartame, dextrose, mannitol, saccharin sodium, sorbitol, sucrose, and their combinations. Sweeteners may be included in amounts ranging from about 0.5% to about 10% by weight of the total composition. These ingredients collectively improve the taste and overall acceptability of the composition, particularly for oral use.

The composition, when formulated for topical application to oral mucosal surface, may include one or more bio-adhesive enhancers to enhance adhesion to the mucosal tissue and improve the permeability of the active ingredients. These enhancers help ensure prolonged contact time at the application site, facilitating effective delivery of the active compounds. Exemplary bio-adhesive enhancers include chitosan and trimethyl chitosan chloride, known for their excellent bio-adhesive properties and ability to enhance permeability. The bio-adhesive enhancers can be incorporated in amounts ranging from about 0.1% to about 8% by weight, based on the total weight of the topical composition.

In some embodiments, the topical composition described herein is formulated to deliver time-controlled, sustained release of the active ingredients over an extended period, thereby offering prolonged pain relief. These formulations include one or more release control agents that modulate the release rate of the active ingredients upon topical application. The controlled release can be triggered, for example, by contact with the skin or mucosal surfaces, ensuring a steady and prolonged release of the active ingredients over an extended period. This mechanism enhances therapeutic efficacy by maintaining optimal concentrations of the active ingredients at the site of application while minimizing the need for frequent reapplication. The release control agents employed in the composition include controlled-release polymers, which play a crucial role in modulating the diffusion or degradation processes that govern the release kinetics. Controlled-release polymers, such as hydroxypropyl cellulose (HPC), hydroxypropyl methyl cellulose (HPMC), polyvinyl alcohol (PVA), Carbopol, and polyethylene oxide (PEO), are particularly advantageous due to their biocompatibility, ease of formulation, and ability to form hydrophilic matrices or gels. These polymers can swell upon contact with moisture, creating a diffusion barrier that slows the release of the active ingredients. For instance, hydroxypropyl methyl cellulose (HPMC) forms a gel layer upon hydration that not only sustains the release of active ingredients but also provides additional benefits, such as enhanced stability and controlled dissolution profiles. Similarly, Carbopol, a synthetic polymer, contributes to the viscosity of the formulation while also acting as a release modulator by forming cross-linked structures that entrap the active ingredients. The choice of controlled-release polymer can be tailored based on the desired release profile, the physicochemical properties of the active ingredients, and the intended application site. These controlled-release polymers may suitably be selected from standard commercially available controlled-release polymers known in the art. The release control agents may be employed in amounts ranging from about 0.1% to 10% by weight, based on the total weight of the topical composition.

In some embodiments, the topical composition disclosed herein includes an encapsulating agent that forms a semi-permeable coating around the active ingredient particles, thereby controlling diffusion and enabling sustained release of the active ingredients. Exemplary encapsulating agents include ethyl cellulose, methyl ethyl cellulose, methyl cellulose, hydroxypropylmethyl cellulose, alginates, and combinations thereof. Preferably, ethyl cellulose is used as the encapsulating agent. The encapsulating agent may be employed in amounts ranging from about 0.1% to 5% by weight, based on the total weight of the topical composition.

In some embodiments, the topical composition disclosed herein enables sustained release of hydroxy-α-sanshool and spilanthol for at least 6 hours, thereby providing pain relief for an equivalent duration.

The pH of the composition is adjusted with a pH adjuster, which can be any suitable pH adjuster. pH adjusters can be either acidic or alkaline. In some embodiments, the pH adjuster can be selected from the group consisting of citric acid, lactic acid, sodium hydroxide, and triethanolamine. The concentration of the pH adjuster can be any concentration suitable for adjusting the composition to a pH of from about 4.5 to about 6.5. In some embodiments, the pH adjuster is included in amounts ranging from about 0.05% to about 5% by weight of the total composition.

Suitable buffers for use in the topical compositions described in the present disclosure are those with sufficient buffering capacity to maintain a pH range of 4.5 to 6.5. Exemplary buffers include, but are not limited to, citric acid, sodium citrate, lactic acid, sodium lactate, succinic acid, sodium succinate, sodium acetate, acetic acid, sodium phosphate, phosphoric acid, sodium ascorbate, tartaric acid, maleic acid, ascorbic acid, sodium bicarbonate, histidine, sodium benzoate, benzoic acid, and combinations thereof. In one embodiment, the buffer is at a concentration between 1 mM and 100 mM. In some embodiments, the pH buffering agent is included in amounts ranging from about 0.05% to about 3% by weight of the total composition.

The topical composition can include water in an amount ranging from about 1% to about 80% by weight, preferably from about 10% to 70% by weight based upon the total weight of the composition.

The composition disclosed herein may be formulated as an ointment, cream, gel, patch, lotion, spray, or foam to suit different application preferences.

In some embodiments, the composition is formulated in the form of cream.

In some embodiments, the composition is formulated in the form of lotion.

In some embodiments, the composition is formulated in the form of gel.

In some embodiments, the composition is formulated as a patch.

In some embodiments, the composition is formulated in the form of oral gel.

The present disclosure is also directed to methods for treating pain by topically administering a composition containing hydroxy-α-sanshool and spilanthol to an area in need of pain relief.

Further embodiments of the present disclosure are provided in the following.

Embodiment 1: A topical composition comprising, based on 100 weight/weight percent, from about 0.001% to about 20% by weight of hydroxy-α-sanshool, from about 0.001% to about 20% by weight of spilanthol, and one or more topically acceptable excipients.

Embodiment 2: The composition of Embodiment 1, wherein the one or more topically acceptable excipients are selected from the group consisting of gel-forming agents, permeation enhancers, viscosity enhancers, mucosal absorption enhancers, solubilizers, stabilizers, antioxidants, hydrating agents, aqueous carriers, preservatives, pH adjusters, pH buffering agents, sweeteners, flavoring agents, bio-adhesive enhancers, encapsulating agents, and release control agents.

Embodiment 3: The composition of any of Embodiments 1-2, wherein the composition is in the form of an ointment, cream, gel, patch, lotion, spray, or foam.

Embodiment 4: A composition for topical application to a mammal's skin for pain relief, the composition comprising, based on 100 weight/weight percent:
from about 0.001% to about 20% by weight of hydroxy-α-sanshool;
from about 0.001% to about 20% by weight of spilanthol;
from about 0.1% to about 10% by weight of one or more gel-forming agents;
from about 0.1% to about 15% by weight of one or more permeation enhancers;
from about 0.5% to about 25% by weight of one or more solubilizers;
from about 1% to about 8% by weight of one or more hydrating agents; and,
from about 1% to about 80% by weight of water, wherein the composition has a pH of 4.5 to 6.5.

Embodiment 5: The composition of Embodiment 4 further comprising:
from about 0.1% to about 5% by weight of one or more antioxidants;
from about 0.1% to about 5% by weight of one or more preservatives;
from about 0.05% to about 5% by weight of a pH adjuster; and, from about 0.05% to about 3% by weight of a pH buffering agent.

Embodiment 6: A composition for topical application to a mammal's oral mucosal surface for treating pain, the composition comprising, based on 100 weight/weight percent:
from about 0.001% to about 20% by weight of hydroxy-α-sanshool;
from about 0.001% to about 20% by weight of spilanthol;
from about 0.1% to about 10% by weight of one or more gel forming agents;
from about 0.1% to about 15% by weight of one or more permeation enhancers;
from about 0.5% to about 25% by weight of one or more solubilizers;
from about 0.1% to about 8% by weight of bio-adhesive enhancers;
from about 0.5% to about 10% by weight of one or more sweeteners;
from about 0.1% to about 5% by weight of one or more flavoring agents;
from about 1% to about 8% by weight of one or more hydrating agents; and,
from about 1% to about 80% by weight of water, wherein the composition has a pH of 4.5 to 6.5.

Embodiment 7: The composition of Embodiment 6 further comprising:
from about 0.1% to about 5% by weight of one or more preservatives;
from about 0.05% to about 5% by weight of a pH adjuster; and, from about 0.05% to about 3% by weight of a pH buffering agent.

Embodiment 8: A composition for topical application to a mammal's skin for the treatment of pain, the composition comprising, based on 100 weight/weight percent:
from about 0.001% to about 20% by weight of hydroxy-α-sanshool;
from about 0.001% to about 20% by weight of spilanthol;
from about 0.1% to about 10% by weight of one or more release control agents;
from about 0.5% to about 5% by weight of an encapsulating agent;
from about 0.1% to about 15% by weight of one or more permeation enhancers;
from about 0.5% to about 25% by weight of one or more solubilizers;
from about 1% to about 8% by weight of one or more hydrating agents; and,
from about 1% to about 80% by weight of water, wherein the composition has a pH of 4.5 to 6.5.

Embodiment 9: The composition of Embodiment 8 further comprising:
from about 0.1% to about 5% by weight of one or more antioxidants;
from about 0.1% to about 5% by weight of one or more preservatives;
from about 0.05% to about 5% by weight of a pH adjuster; and, from about 0.05% to about 3% by weight of a pH buffering agent.

Embodiment 10: A method for treating pain by topically administering a composition containing hydroxy-α-sanshool and spilanthol to an area in need of pain relief.

### EXAMPLES

The present disclosure is further explained in the form of following examples. However, it is to be understood that the following examples are merely illustrative and are not to be taken as limitations upon the scope of the invention. Various changes and modifications to the disclosed embodiments will be apparent to those skilled in the art. Such changes and modifications may be made without departing from the scope of the invention. The appropriate procedure to prepare topical gel composition will be apparent to those skilled in the art.

### Example 1: Preparation of Gel for Topical Application to the Skin

A gel was prepared according to the ingredients and amounts shown in Table 1.

TABLE 1

| | | Amount |
|---|---|---|
| Ingredient | Role | (wt. %) |
| Hydroxy-α-sanshool | Active ingredient | 0.001-20% |
| Spilanthol | Active ingredient | 0.001-20% |
| Carbomer | Gel-forming agent | 0.5-2% |
| Propylene glycol | Permeation enhancer | 5-10% |
| Ethanol | Solubilizer | 5-10% |
| Tocopherol | Antioxidant | 0.1-0.5% |
| Sodium metabisulfite | Antioxidant | 0.1-0.5% |
| Glycerin | Hydrating agent | 2-5% |
| Phenoxyethanol | Preservative | 0.5-1% |
| Triethanolamine | pH adjuster | 0.01-10% |
| Citrate buffer | pH buffering agent | 0.01-10% |
| Water | Carrier | Q.S. |

The active ingredients, spilanthol and hydroxy-α-sanshool, were dissolved in ethanol to prepare a solution. Separately, carbomer was hydrated in purified water to form a gel base. The ethanol solution containing the active ingredients was then gradually added to the gel base with continuous stirring to ensure uniform incorporation. Propylene glycol, glycerin, tocopherol, sodium metabisulfite, and phenoxyethanol were sequentially added to the mixture. The pH of the formulation was adjusted to a range of 5 to 6 using triethanolamine to achieve the desired skin compatibility. Finally, citrate buffer was added to complete the gel formulation. The resulting gel composition demonstrated controlled release, with the carbomer gel matrix ensuring sustained release of spilanthol and hydroxy-α-sanshool. Propylene glycol enhanced permeability, facilitating effective delivery of the active ingredients to target sites through the skin layers. Antioxidants and pH buffering agents contributed to the stability of the composition by minimizing degradation of the active compounds. Additionally, glycerin helped maintain skin hydration and prevented irritation, ensuring optimal skin compatibility.

### Example 2: Preparation of Oral Gel for Topical Application to Oral Mucosa

An oral gel was prepared according to the ingredients and amounts shown in Table 2.

TABLE 2

| | | Amount |
|---|---|---|
| Ingredient | Role | (wt. %) |
| Hydroxy-α-sanshool | Active ingredient | 0.001-20% |
| Spilanthol | Active ingredient | 0.001-20% |
| Carbomer ® 934P | Gel-forming agent | 0.5-1.5% |
| HPMC | Gel-forming agent | 1-3% |
| Propylene glycol | Permeation enhancer | 5-10% |
| Oleic acid | Permeation enhancer | 0.5-1% |
| Polysorbate 80 | Solubilizer | 0.5-1% |
| Chitosan | Bio-adhesive enhancer | 0.5-1% |
| Xylitol | Sweetener | 1-2% |
| Peppermint oil | Flavoring agent | 0.1-0.5% |
| Glycerin | Hydrating agent | 2-5% |
| Potassium sorbate | Preservative | 0.1-0.2% |
| Citric acid | pH adjuster | 0.01-10% |
| Sodium citrate buffer | pH buffering agent | 0.01-10% |
| Water | Carrier | Q.S. |

The active ingredients, spilanthol and hydroxy-α-sanshool, were dissolved in a mixture of propylene glycol, oleic acid, and polysorbate 80. Separately, carbomer and HPMC were hydrated in purified water to create a gel base. The solution containing the active ingredients was gradually incorporated into the gel base with continuous stirring to ensure uniform distribution. Subsequently, glycerin, xylitol, peppermint oil, potassium sorbate, and chitosan were sequentially added to the mixture. The pH of the formulation was adjusted to a range of 5.5 to 6.5 using citric acid. Finally, citrate buffer was added to complete the preparation of the oral gel formulation. The resulting oral gel exhibited excellent bioadhesion, with the combination of carbomer, HPMC, and chitosan ensuring prolonged contact with the buccal mucosa to enhance absorption. Propylene glycol and oleic acid significantly improved the permeability of spilanthol and hydroxy-α-sanshool through the mucosal layer. The pH-adjusted, buffered gel formulation provided stability and minimized irritation. Furthermore, the inclusion of sweeteners and flavoring agents enhanced user acceptability by improving the taste, making the gel more tolerable for oral application.

### Example 3: Preparation of Controlled-Release Gel for Topical Application to the Skin

A controlled-release gel was prepared according to the ingredients and amounts shown in Table 3.

TABLE 3

| | | Amount |
|---|---|---|
| Ingredient | Role | (wt. %) |
| Hydroxy-α-sanshool | Active ingredient | 0.001-20% |
| Spilanthol | Active ingredient | 0.001-20% |
| Carbopol ® 934P | Release control agent | 0.5-1.5% |
| HPMC | Release control agent | 2-4% |
| Ethyl cellulose | Encapsulating agent | 1-2% |
| Propylene glycol | Permeation enhancer | 5-10% |
| Oleic acid | Permeation enhancer | 0.5-1% |
| Polysorbate 80 | Solubilizer | 0.5-1% |
| Glycerin | Hydrating agent | 2-5% |
| Tocopherol | Antioxidant | 0.1-0.5% |
| Sodium metabisulfite | Antioxidant | 0.1-0.5% |
| Phenoxyethanol | Preservative | 0.5-1% |
| Triethanolamine | pH adjuster | 0.01-10% |
| Citrate buffer | pH buffering agent | 0.01-10% |
| Water | Carrier | Q.S. |

The active ingredients, spilanthol and hydroxy-α-sanshool, were dissolved in a mixture of propylene glycol, oleic acid, and polysorbate 80. Separately, Carbopol and HPMC were hydrated in purified water to create a gel base. The solution containing the active ingredients was gradually incorporated into the gel base with continuous stirring to ensure uniform distribution. Then, ethyl cellulose (pre-dispersed in a small amount of ethanol) was added to the mixture to encapsulate the active ingredients. Subsequently, glycerin, tocopherol, sodium metabisulfite, and phenoxyethanol were sequentially added to the mixture. The pH of the formulation was adjusted to a range of 5 to 6 using triethanolamine to achieve the desired skin compatibility. Finally, citrate buffer was added to complete the gel formulation. The resulting gel formulation demonstrated prolonged activity, with the combination of Carbopol, HPMC, and ethyl cellulose ensuring the sustained release of spilanthol and hydroxy-α-sanshool for over 6 hours. The pH-adjusted formulation enhanced skin compatibility, minimizing irritation while the inclusion of moisturizers and antioxidants improved user comfort. Additionally, the gel matrix provided stability and preserved the integrity of the active compounds during storage and application.

### Example 4: Skin Topical Gel Formulation

A skin topical gel formulation was prepared with the following composition by weight percentage as shown in table 4:

TABLE 4

| Ingredient | Role | % (w/w) |
|---|---|---|
| Hydroxy-α-sanshool | Active ingredient | 2.0% |
| Spilanthol | Active ingredient | 2.0% |
| Carbomer (polyacrylate) | Gel-forming agent | 1.0% |
| Propylene glycol | Permeation enhancer | 8.0% |
| Ethanol | Solubilizer (co-solvent) | 8.0% |
| Tocopherol (Vitamin E) | Antioxidant | 0.2% |
| Sodium metabisulfite | Antioxidant | 0.1% |
| Glycerin | Hydrating agent | 4.0% |
| Phenoxyethanol | Preservative | 0.8% |
| Triethanolamine (TEA) | pH adjuster (neutralizer) | 0.5% |
| Citrate buffer (pH 5-6) | pH buffering agent | 1.0% |
| Purified water | Aqueous carrier (gel base) | 72.4% |
| | Total | 100% |

Hydroxy-α-sanshool and spilanthol, derived from plant sources, serve as the primary active pharmaceutical ingredients, each providing analgesic and anti-inflammatory effects for pain management, toothache relief, and chronic pain conditions.

Carbomer polyacrylate forms the gel matrix, providing a semi-solid consistency and establishing the pharmaceutical base. Propylene glycol (8% w/w) and ethanol (8% w/w) function synergistically as permeation enhancers and solubilizers for the lipophilic active compounds, improving their bioavailability and transepidermal penetration.

The dual antioxidant system comprises tocopherol (vitamin E, 0.2% w/w) and sodium metabisulfite (0.1% w/w), which protect the sensitive alkamide actives from oxidative degradation during manufacturing, storage, and application. Glycerin (4.0% w/w) functions as a hydrating agent (humectant), maintaining skin moisture and enhancing the sensorial properties of the formulation. Phenoxyethanol (0.8% w/w) serves as the antimicrobial preservative, inhibiting microbial growth throughout the shelf-life.

Triethanolamine (0.5% w/w) neutralizes the carbomer polymer, adjusting the pH to approximately 5.5, thereby achieving optimal gel viscosity and consistency. A citrate buffer system (1.0% w/w) maintains pH stability between 5 and 6, ensuring carbomer functionality is preserved and preventing degradation of the active compounds over time. Purified water (72.4% w/w) serves as the aqueous vehicle and primary carrier for all dissolved and dispersed components.

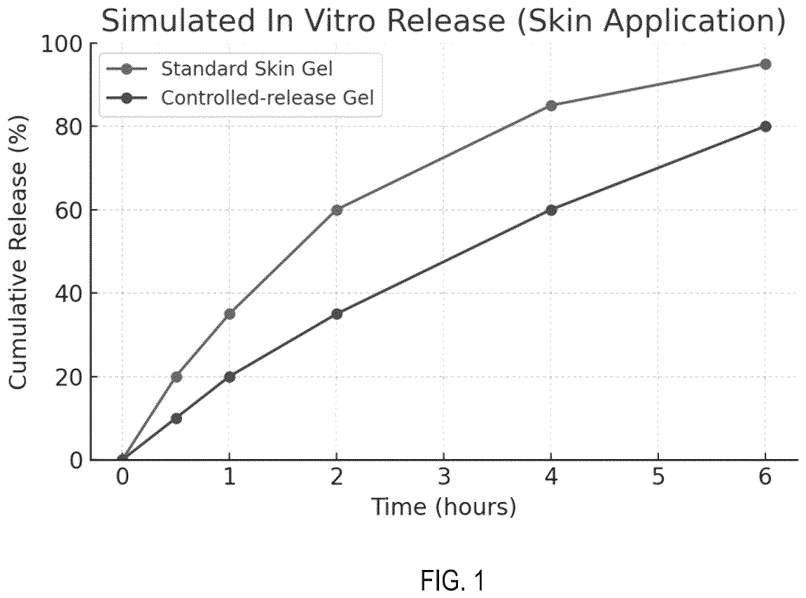

In Vitro Release Profile: The skin gel formulation exhibits a controlled, biphasic release profile of the active compounds over a 6-hour period (see FIG. 1). The formulation demonstrates approximately 35% release of spilanthol and hydroxy-α-sanshool by 1 hour, with cumulative release reaching approximately 95% by 6 hours, thereby balancing immediate therapeutic onset with sustained delivery.

The carbomer gel matrix provides a diffusional barrier that sustains the release of the actives, preventing burst release while maintaining therapeutic plasma levels over the application period. Propylene glycol's inclusion as a penetration enhancer accounts for the initial rapid release phase (approximately 20-30% in the first hour), reducing stratum corneum resistance and facilitating early diffusion of actives through the gel into the dermal layers. Beyond this initial phase, the cross-linked carbomer network slows further diffusion, yielding a controlled, gradual release profile over the subsequent 4-6 hours.

Mechanistically, propylene glycol enhances skin permeability by disrupting the lipid matrix of the stratum corneum, improving penetration of the alkamide actives. Glycerin's humectant effect maintains hydration of the stratum corneum, further improving dermal uptake and bioavailability. This formulation thereby balances immediate pain relief via the initial release phase with sustained therapeutic action via the gel matrix diffusion barrier.

Stability Data: The formulation exhibits excellent chemical and physical stability under typical storage conditions. Accelerated aging studies (40 °C for 3 months) demonstrate less than approximately 5-10% loss of active content, with pH remaining in the optimal 5-6 range. The citrate buffer system prevents any significant pH drift during accelerated aging. Viscosity shows minimal change (less than approximately 10%) after high-temperature storage, with the carbomer gel network remaining intact.

Long-term storage at 25 °C for 12 months is projected to have negligible impact on product performance, with the gel remaining homogenous and effective. The antioxidant system protects against degradation of spilanthol and hydroxy-α-sanshool during storage, and the preservative system prevents any microbial contamination. No phase separation or precipitation is expected, as the ethanol co-solvent ensures miscibility and the actives remain solubilized by propylene glycol within the gel matrix. Based on the formulation's design incorporating preservatives, antioxidants, and pH buffering agents, a shelf-life of at least 2 years under normal storage conditions is projected, maintaining clarity, pH stability, and analgesic efficacy.

### Example 5: Oral Topical Gel Formulation

An alternative topical gel formulation was prepared for intraoral (oral mucosal) application, with modifications to enhance tolerability and address specific requirements for oral use as shown in table 5:

TABLE 5

| Ingredient | Role | % (w/w) |
|---|---|---|
| Hydroxy-α-sanshool | Active ingredient | 2.0% |
| Spilanthol | Active ingredient | 2.0% |
| Carbomer 934P | Gel-forming agent | 1.0% |
| Hydroxypropyl | Gel-forming/bioadhesive | 2.0% |
| methylcellulose (HPMC) | polymer | |
| Propylene glycol | Permeation enhancer | 8.0% |
| Oleic acid | Penetration enhancer | 1.0% |
| Polysorbate 80 | Solubilizer (surfactant) | 0.5% |
| Chitosan | Bio-adhesive enhancer | 1.0% |
| Xylitol | Sweetener (taste agent) | 2.0% |
| Peppermint oil | Flavoring agent | 0.2% |
| Glycerin | Humectant (hydration) | 4.0% |
| Potassium sorbate | Preservative | 0.2% |
| Citric acid | pH adjuster (acidulant) | 0.1% |
| Sodium citrate buffer | pH buffering agent | 1.0% |
| Purified water | Aqueous gel base (carrier) | 75.0% |
| | Total | 100% |

The oral formulation reduces the concentration of hydroxy-α-sanshool and spilanthol to 1.5% w/w each, compared to 2.0% in the dermal formulation, ensuring tolerability for intraoral application. Oleic acid (2.0% w/w) replaces a portion of propylene glycol, enhancing permeability of the alkamide actives across oral mucosa while providing improved tolerability. The carbomer concentration is reduced to 0.8% w/w to achieve a softer gel consistency suitable for oral application, while maintaining sufficient matrix viscosity for sustained release.

The oral formulation retains the dual antioxidant system, preservative, and pH buffering system, ensuring chemical stability and antimicrobial efficacy equivalent to the dermal formulation. The increased glycerin concentration (5.0% w/w) provides enhanced lubrication and improved sensorial properties for oral application. The formulation achieves sustained release of actives over 4-6 hours while remaining non-irritating to oral tissues.

Simulated In Vitro Release Profile: In a mucosal in vitro model, for example a Franz diffusion cell equipped with buccal tissue, the oral gel of Example 2 exhibits a sustained yet substantial release of the active ingredients over a period of 6 hours. A simulated 6-hour in vitro release profile demonstrates that approximately 30% of the total amount of hydroxy-α-sanshool and spilanthol is released by about 1 hour, and about 90% is released by about 6 hours, indicating a balance between bioadhesive retention of the gel and continuous release of the actives.

The initial release during the first hour, which is in the range of about 25-30%, provides a prompt numbing effect suitable for acute oral pain relief. This early phase is facilitated by the presence of propylene glycol and oleic acid, which act as permeation enhancers and promote rapid flux of the active ingredients into the mucosal tissue. Following this initial phase, the bioadhesive polymer matrix comprising carbomer, hydroxypropyl methylcellulose (HPMC), and chitosan hydrates and swells at the application site, thereby forming a diffusional barrier that prolongs release of the actives.

Upon contact with saliva, HPMC and carbomer form a viscoelastic hydrophilic gel layer that slows further diffusion of the active ingredients, consistent with sustained-release mechanisms observed in hydrophilic matrix systems. Chitosan contributes additional mucoadhesion and may transiently open tight junctions in the mucosa, which can enhance transmucosal permeability. The combined action of carbomer, HPMC, and chitosan maintains the gel at the site of application and provides gradual release of hydroxy-α-sanshool and spilanthol, thereby affording an extended window of analgesia in the oral cavity. Concurrently, propylene glycol and oleic acid disrupt the mucosal lipid barrier to facilitate absorption of the released actives, ensuring that sufficient quantities permeate to the target tissues despite the controlled-release behavior. As a result, the oral gel adheres to the mucosa and provides pain relief for several hours without requiring frequent reapplication.

Stability Data: The oral mucosal gel of Example 2 is formulated to remain chemically and physically stable under both normal and accelerated storage conditions. The inclusion of a citrate buffer system and a preservative (potassium sorbate) provides resistance to microbial contamination and helps minimize degradation of the active ingredients. Under accelerated stability testing conditions, for example at about 40 °C and elevated humidity, only minor changes are expected: the pH may decrease slightly due to the presence of citric acid; however, the buffer maintains the pH within an acceptable range, and a modest decrease in viscosity may occur over time.

HPMC and chitosan are generally stable polymers, although prolonged exposure to high temperature can result in some reduction of molecular weight, potentially leading to a small decrease in gel viscosity. In view of the protective polymer matrix, any viscosity loss after 3 months at 40 °C is expected to remain within approximately 10-15% of the initial value. The active alkamides exhibit some susceptibility to oxidation, but protection is afforded by antioxidant components (for example, tocopherol, if included as in the skin gel embodiments) and by the mildly acidic pH of the formulation. Peppermint oil, being volatile, may show a slight reduction in flavor intensity under extended high-temperature exposure; however, under typical room-temperature storage this effect is negligible.

Long-term stability at ambient temperature is expected to be excellent: after about 12 months of storage at room temperature, the gel is projected to retain at least about 90% of the initial active content without significant discoloration or odor change. The mucoadhesive properties derived from chitosan and HPMC remain substantially unchanged, as these polymers do not rapidly degrade under normal conditions. In combination, the preservative system and reduced water activity (due to polyols such as glycerin and propylene glycol) inhibit microbial growth and support product integrity. When packaged in an airtight container, such as an aluminum or laminated tube that limits evaporation of volatile components, the oral gel maintains its efficacy, organoleptic properties, and texture throughout its intended shelf life, enabling consistent dosing and flavor upon use.

### Example 6: Controlled-Release Gel Formulation

A controlled-release gel formulation was prepared with the following composition by weight percentage, as shown in table 6:

TABLE 6

| Ingredient | Role | % (w/w) |
|---|---|---|
| Hydroxy-α-sanshool | Active ingredient | 2.0% |
| Spilanthol | Active ingredient | 2.0% |
| Carbopol 934P (Carbomer) | Gel matrix/release | 1.0% |
| | controller | |
| Hydroxypropyl | Release control | 3.0% |
| methylcellulose (HPMC) | polymer | |
| Ethyl cellulose | Encapsulating agent | 1.5% |
| | (insoluble polymer) | |
| Propylene glycol | Permeation enhancer | 8.0% |
| Oleic acid | Permeation enhancer | 1.0% |
| Polysorbate 80 | Solubilizer | 0.5% |
| | (surfactant) | |
| Glycerin | Hydrating agent | 4.0% |
| Tocopherol (Vitamin E) | Antioxidant | 0.2% |
| Sodium metabisulfite | Antioxidant | 0.1% |
| Phenoxyethanol | Preservative | 0.8% |
| Triethanolamine | pH adjuster (gel | 0.5% |
| | neutralizer) | |
| Citrate buffer (pH ~5.5) | pH buffering agent | 1.0% |
| Purified water | Carrier (gel base) | 74.4% |
| | Total | 100% |

This formulation builds upon the standard skin gel by incorporating HPMC and ethyl cellulose as additional controlled-release polymers. Carbopol and HPMC together form a swellable gel matrix that controls diffusion of the active ingredients, while ethyl cellulose, being water-insoluble, encapsulates or associates with the drug molecules to further delay their release. Propylene glycol and oleic acid are retained to ensure that, once released from the gel matrix, the active compounds can effectively permeate the skin. The remaining excipients correspond to those of the standard gel and serve to provide skin hydration, antioxidant protection, preservation, and pH control for optimal carbomer performance and dermal compatibility.

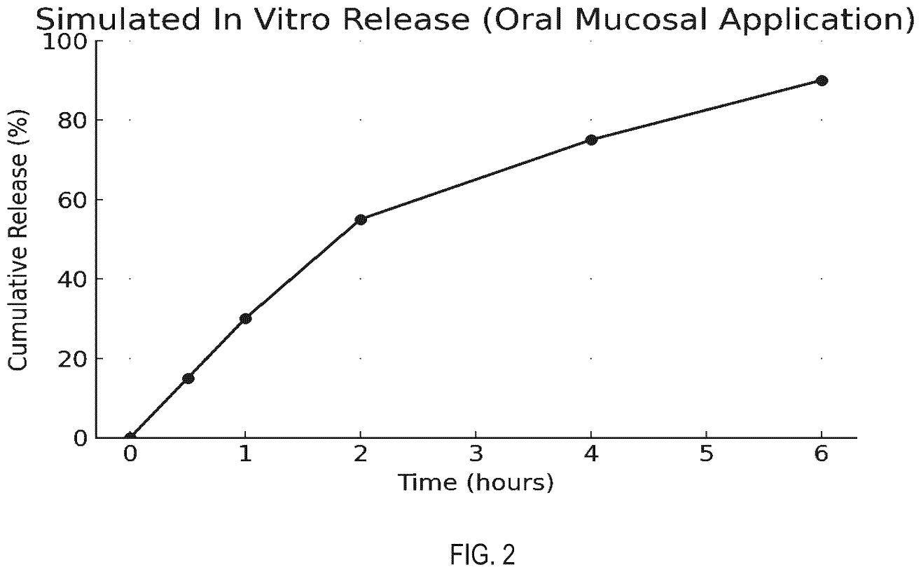

Simulated In Vitro Release Profile: The controlled-release gel of Example 6 is designed to extend delivery of hydroxy-α-sanshool and spilanthol relative to the standard gel formulation. In a 6-hour in vitro skin permeation test (see FIG. 2), the controlled-release gel releases only about 10% of the active ingredients within the first 30 minutes and about 20% by 1 hour, compared to approximately 35% release from the standard gel over the same period. The release then proceeds gradually, reaching about 50-60% by 4 hours and approximately 80% by 6 hours, with additional release continuing beyond 6 hours.

This pronounced reduction in initial release rate is attributed to the multi-polymer matrix of carbopol, HPMC, and ethyl cellulose. Upon application to the skin and subsequent hydration, carbopol and HPMC rapidly swell to form a thick hydrophilic gel layer that retards diffusion of the active ingredients. HPMC in particular forms a viscous hydrated barrier that the actives must traverse slowly, thereby contributing significantly to sustained release. Ethyl cellulose functions as an encapsulating or dispersing water-insoluble phase, such that a fraction of the sanshool and spilanthol is released only as this ethyl cellulose domain gradually permits diffusion or erosion, producing a prolonged release “tail.”

Mechanistically, the combination of a swellable hydrophilic matrix (carbopol/HPMC) with a water-insoluble diffusional barrier (ethyl cellulose) provides dual control over drug release. An initial, relatively small release of any unencapsulated drug located near the gel surface may occur, but thereafter the hydrated gel network and ethyl cellulose phase maintain a near-linear sustained-release profile. Throughout the test period, propylene glycol and oleic acid act as permeation enhancers to promote dermal uptake of the fraction of actives that is released, so therapeutic levels in the skin are maintained even at reduced release rates. Overall, the controlled-release gel delivers prolonged analgesic activity from a single application, thereby reducing the need for frequent reapplication.

Stability Data: The controlled-release gel of Example 3 is formulated to exhibit stability characteristics comparable to those of the standard gel, while incorporating additional polymers that are themselves stable under typical storage conditions. Accelerated stability studies are expected to show that the formulation remains physically uniform, with an initial slight increase in viscosity over the first several days post-manufacture as HPMC fully hydrates, followed by stable rheological behavior thereafter.

At elevated temperature (for example 40 °C), carbopol and HPMC may experience minor reductions in viscosity over time; however, their combined presence supports the gel network and helps maintain overall structural integrity. Ethyl cellulose, being chemically inert and water-insoluble, is highly stable and provides an additional protective microenvironment for a portion of the active ingredients, since drug associated with ethyl cellulose is less exposed to oxygen and water until released. The antioxidants (tocopherol and sodium metabisulfite) minimize oxidative degradation of the alkamide actives such that, even after 3 months at 40 °C, the loss of sanshool and spilanthol potency is projected to be low, for example on the order of about 5-7%.

The citrate buffer and appropriate neutralization maintain the pH in the range of about 5.0-5.5, which is suitable for carbomer gel formation and is not expected to drift substantially during storage. No significant changes in color or odor are anticipated; the product remains an opaque or translucent gel with acceptable cosmetic characteristics over time. Over 12-24 months of storage at room temperature, the controlled-release gel is expected to retain its extended-release performance, as the HPMC and ethyl cellulose polymers do not undergo substantial degradation under normal storage conditions. Thus, batches stored for one year or longer provide a release profile comparable to that of freshly manufactured product. The design of the controlled-release gel therefore not only prolongs drug delivery during use, but also contributes to stability by physically protecting the actives and maintaining a buffered, antioxidant-containing hydrated matrix throughout the product's shelf life.

The foregoing examples are illustrative of specific embodiments encompassed by the present disclosure. The scope of the invention is defined by the appended claims and includes all compositions and methods within the scope of such claims, as well as variations and modifications apparent to those skilled in the pharmaceutical formulation arts.

It will be understood that variations in component concentrations, substitution of functionally equivalent excipients, and modifications of processing parameters remain within the scope of this disclosure, provided that the resulting formulation exhibits the analgesic and anti-inflammatory properties, skin penetration enhancement, and chemical stability characteristics disclosed herein.

What is claimed is:

1. A topical composition for treating pain and inflammation, based on the total weight of the composition, wherein the composition comprises:
(a) from about 0.001 wt % to about 20 wt % hydroxy-α-sanshool based on the total weight of the composition;
(b) from about 0.001 wt % to about 20 wt % spilanthol based on the total weight of the composition; and,
(c) from about 5 wt % to about 95 wt % of one or more topically acceptable excipients based on the total weight of the composition.

2. The composition of claim 1, wherein the one or more topically acceptable excipients comprise gel-forming agents, permeation enhancers, solubilizers, antioxidants, hydrating agents, aqueous carriers, preservatives, pH adjusters, pH buffering agents, sweeteners, flavoring agents, bio-adhesive enhancers, encapsulating agents, or release control agents.

3. The composition of claim 2, wherein the gel-forming agents comprise polyacrylates, cellulose derivatives, polyvinyl alcohols, polyvinyl acetate, or mixtures thereof.

4. The composition of claim 2, wherein the permeation enhancers comprise glycols, polyols, fatty acids, ether alcohols, cyclic ether alcohols, alkoxy substituted cyclic ethers, fatty alcohol esters, or mixtures thereof.

5. The composition of claim 2, wherein the solubilizer comprises ethanol, Polysorbate 80, or mixtures thereof.

6. The composition of claim 2, wherein the antioxidants comprise tocopherol, butyl hydroxytoluene, ascorbyl palmitate, propyl gallate, sodium metabisulfite, potassium metabisulfite or mixtures thereof.

7. The composition of claim 2, wherein the antioxidants comprise hydrating agents glycerin, hyaluronic acid, petrolatum, cetostearyl alcohol or mixtures thereof.

8. The composition of claim 2, wherein the aqueous carriers is water.

9. The composition of claim 2, wherein the preservatives comprise phenoxyethanol, parabens, sodium benzoate, potassium sorbate, thimerosal, chlorobutanol, benzalkonium chloride, benzyl alcohol or mixtures thereof.

10. The composition of claim 2, wherein the pH adjusters comprise citric acid, lactic acid, sodium hydroxide, and triethanolamine; or the pH buffering agents is selected from the group consisting of citric acid, sodium citrate, lactic acid, sodium lactate, succinic acid, sodium succinate, sodium acetate, acetic acid, sodium phosphate, phosphoric acid, sodium ascorbate, tartaric acid, maleic acid, ascorbic acid, sodium bicarbonate, histidine, sodium benzoate, benzoic acid, or combinations thereof.

11. The composition of claim 2, wherein the sweeteners comprise xylitol, aspartame, dextrose, mannitol, saccharin sodium, sorbitol, sucrose, and combinations thereof; or the flavoring agent is selected from the group consisting of peppermint oil, anise oil, cinnamon oil, cocoa, menthol, orange oil, vanillin or combination thereof.

12. The composition of claim 2, wherein the bio-adhesive enhancers are chitosan or trimethyl chitosan chloride.

13. The composition of claim 2, wherein the encapsulating agents comprise ethyl cellulose, methyl ethyl cellulose, methyl cellulose, hydroxypropylmethyl cellulose, alginates, or combinations thereof.

14. The composition of claim 2, wherein the release control agents comprise hydroxypropyl cellulose (HPC), hydroxypropyl methyl cellulose (HPMC), polyvinyl alcohol (PVA), Carbopol, polyethylene oxide (PEO), or combination thereof.

15. The composition of claim 1, wherein the composition is in the form of ointment, cream, gel, patch, lotion, spray, or foam.

16. The composition of claim 1, wherein the composition is a controlled release formulation, or sustained release formulation.

17. The composition of claim 1, wherein the composition is a skin topical gel formulation or oral mucosal gel formulation.

18. A method of treating a pain or inflammation comprising administering a therapeutically effective amount of the composition of claim 1 to a subject in need thereof.

19. The composition of claim 1, based on the total weight of the composition, wherein the composition comprises:
(a) from about 0.001 wt % to about 20 wt % hydroxy-α-sanshool;
(b) from about 0.001 wt % to about 20 wt % spilanthol;
(c) from about 0.1 wt % to about 10 wt % of one or more gel-forming agents;
(d) from about 0.1 wt % to about 15 wt % of one or more permeation enhancers;
(e) from about 0.5 wt % to about 25 wt % of one or more solubilizers;
(f) from about 1 wt % to about 8 wt % of one or more hydrating agents;
(g) from about 0.1 wt % to about 5 wt % of one or more preservatives;
(h) from about 0.05 wt % to about 5 wt % of a pH adjuster;
(i) from about 0.05 wt % to about 3 wt % of a pH buffering agent; and,
(j) from about 1 wt % to about 80 wt % water.

20. The composition of claim 19, based on the total weight of the composition, wherein the composition optionally further comprises:
(a) from about 0.1 wt % to about 5 wt % of one or more antioxidants;
(b) from about 0.5 wt % to about 5 wt % of an encapsulating agent;
(c) from about 0.1 wt % to about 10 wt % of one or more release control agents;
(d) from about 0.1 wt % to about 8 wt % of bio-adhesive enhancers;
(e) from about 0.5 wt % to about 10 wt % of one or more sweeteners; or,
(f) from about 0.1 wt % to about 5 wt % of one or more flavoring agents.
